(19) United States
(12) Plant Patent Application Publication
(10) Pub. No.: US 2008/0235840 P1
(43) Pub. Date: Sep. 25, 2008

(54) Liriope muscari plant named 'LIRJ'

(76) Inventor: Todd Anthony Layt, Richmond (AU)

Correspondence Address: Sam R. McCoy Jr., P.O. Box 2108, Mount Pleasant, SC, 29465, US

(21) Appl. No.: 12/077,555

(22) Filed: Mar. 20, 2008

Related U.S. Application Data
(60) Provisional application No. 60/919,799, filed on Mar. 23, 2007.

(51) Int. Cl. A01H 5/00

(52) U.S. Cl. PLT/450

(57) ABSTRACT

‘LIRJ’ is a distinctive variety of Liriope muscari which is characterized by the combination of medium-tall plant height combined with narrow-medium leaf width, very thick leaf thickness, dense foliage and a purple violet flower color.

### LATIN NAME OF THE GENUS AND SPECIES

The Latin name of the genus and species of the novel variety disclosed herein is Liriope muscari.

### VARIETY DENOMINATION

The inventive variety of Liriope disclosed herein has been given the variety denomination ‘LIRJ’.

### BACKGROUND OF THE INVENTION

The present invention relates to a new and distinct perennial variety of Liriope muscari, which has been given the variety denomination of ‘LIRJ’. Its market class is that of an ornamental plant. ‘LIRJ’ is intended for use in landscaping and as a decorative plant.

The Liriope muscari variety ‘LIRJ’ was finally selected in 2002 in an Australian nursery in the state of New South Wales following a selection process carried out from 1999 to 2002 involving approximately 30000 seedlings of Liriope muscari. ‘LIRJ’ is a seedling selection from open pollinated Liriope muscari. ‘LIRJ’ was selected due to its medium-tall plant height combined with narrow-medium leaf width and dense foliage. ‘LIRJ’ was first propagated asexually by division in the state of New South Wales, Australia and has since been asexually propagated by division and micropropagation. The distinctive characteristics of the inventive ‘LIRJ’ variety are stable from generation to generation; clones of the variety produced by asexual reproduction maintain the distinguishing characteristics of the original plant.

‘LIRJ’ has an upright growth habit with a very strong shoot density whereas Liriope muscari has an upright to semi upright growth habit, tending to spread more, with a medium shoot density. ‘LIRJ’ has a tall plant height (60-65 cm in Sydney, NSW Australia) whereas Liriope muscari has a medium plant height (40-50 cm). ‘LIRJ’ has a narrow-medium leaf width (mean 7.5 mm in Sydney, NSW Australia) whereas Liriope muscari has a broad leaf width (mean 8 mm). ‘LIRJ’ has a thick leaf (mean 0.80 mm) whereas Liriope muscari has a medium leaf thickness (mean approximately 0.60 mm).

An application for plant breeders' rights for variety ‘LIRJ’ has been lodged with the Australian Plant Breeders' Rights Office, and was first gazefted in 27 March 2006 under Application No. 2006/037.

### SUMMARY OF THE INVENTION

‘LIRJ’ is a distinctive variety of Liriope muscari which is characterized by the combination of its medium-tall plant height combined with narrow-medium leaf width, broad leaf thickness, dense foliage and a purple violet flower color corresponding to RHS 82B.

### BRIEF DESCRIPTION OF THE FIGURES

FIG. 1 shows a ‘LIRJ’ plant.

### BOTANICAL DESCRIPTION OF THE PLANT

The following is a detailed botanical description of a new and distinct variety of a Liriope muscari ornamental plant known as ‘LIRJ’. Plant observations were made on plants grown in New South Wales, Australia. Unless indicated otherwise, the descriptions disclosed herein are based upon observations made in February 2006 of mature ‘LIRJ’ plants grown in nursery pots.

Those skilled in the art will appreciate that certain characteristics will vary with older or, conversely, younger plants. ‘LIRJ’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 1995 edition. Note that generic color descriptions such as ‘pink’ do not exist in the RHS charts and ‘purple’ color may be referred to more specifically by the corresponding RHS colors quoted.

‘LIRJ’ is a perennial Liriope muscari plant which is a seedling selection from Liriope muscari. After its selection, ‘LIRJ’ was asexually propagated by division and then by micropropagation. ‘LIRJ’ has a green leaf color corresponding to yellow green (RHS 146A) and purple violet flowers (flower color corresponding to RHS 82B) displayed within the foliage.

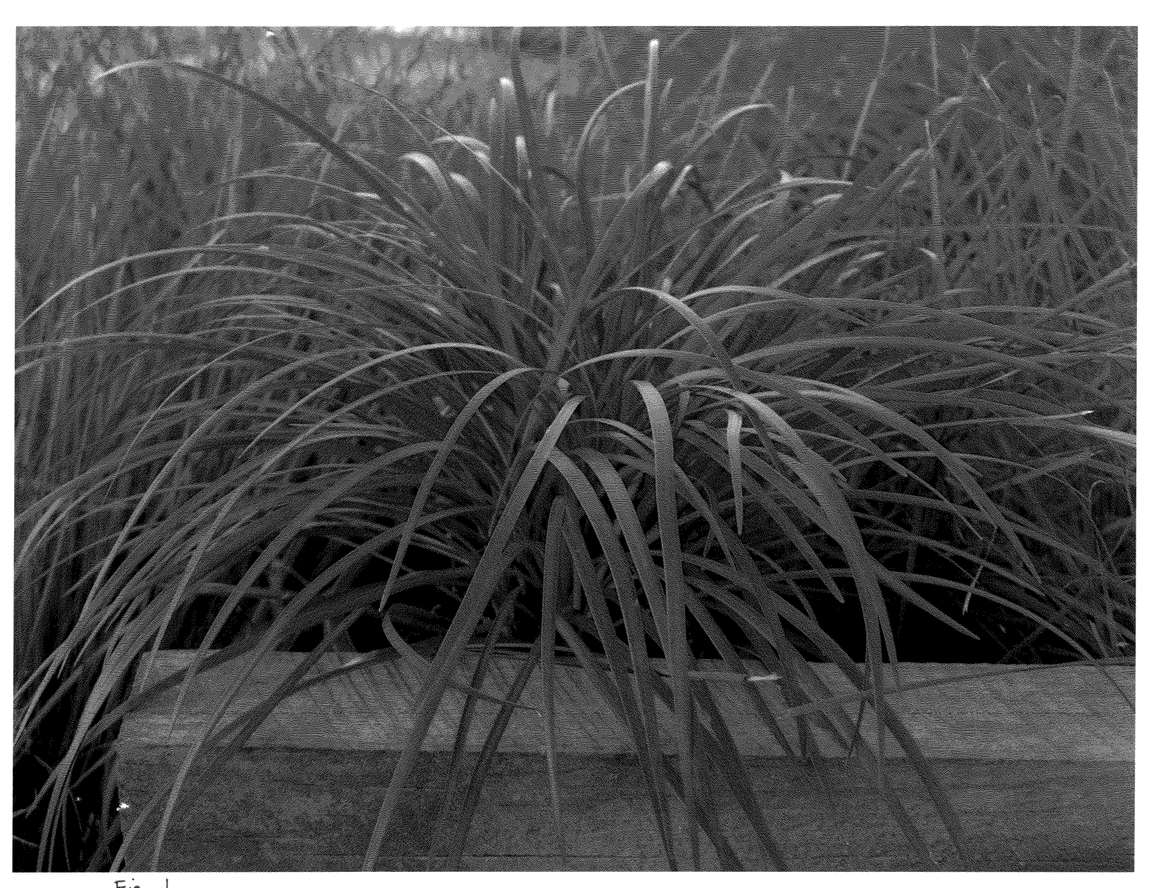

These features and other characteristics are apparent from the description provided below.
Growth habit, dimensions and color: ‘LIRJ’ is a medium-tall plant forming an upright clump with slightly spreading leaves and arching and weeping leaves as the foliage matures. Average plant height is to 60-65 cm and average plant spread is to 90-95 cm in a mature plant grown in a garden in Sydney, New South Wales, Australia (observed January 2007). A representative ‘LIRJ’ plant is shown in FIG. 1. The upper side of the mature leaf is yellow green in color (observed January 2007) corresponding to approximately RHS 146A. Leaf margins and mid ribs do not contrast prominently with the leaf blade. The leaf lower side color corresponds to the upper leaf side but has a less glossy surface texture. The leaf width is narrow-medium (mean 7.5 mm), and reaches an average length of 60 cm in Sydney, NSW, Australia. The leaf thickness (mean 0.80 mm) is greater than usual. The leaf shape is ligulate-ensiform, leaf base is cauline, leaf margin is entire, leaf apex is acute and leaf surface texture is glossy and glabrous. The leaf venation pattern is parallel; the color is the same as the rest of the leaf. The leaf attitude is erect to semi erect with arching and weeping of foliage at maturity.
Roots: The roots of ‘LIRJ’ are colored white (approximately RHS 155C) and fleshy and thick, similar to other Liriope muscari.
Inflorescence: The inflorescence is a raceme with flowers borne in clusters of typically 3. Each flower cluster consists of pedicellate flowers ranging from very a short pedicel length (almost sessile; less than 1 mm) to up to 5 mm long. Inflorescence length is approximately 20-25 cm on average and the raceme position is within the foliage at anthesis. Raceme length is 11-15 cm on average. Peduncle color is grayed purple (RHS 183A-B) mixed with yellow green (approximately RHS 146C-D) creating an overall purple appearance. Each flower cluster is subtended by a small bract colored yellow green (approximately RHS 146C). Pedicel color is initially white (RHS 155C) changing to purple violet (RHS 82D) as the flower opens. The flowering season in Sydney, NSW, Australia is early December until Late February.
Flowers: Flower bud color is yellow (RHS 11D) (immature) and purple violet (RHS 82C-D) just prior to opening. Mature petal color is purple violet (RHS 82B). Anthers are yellow RHS 6B. Flower diameter is approximately 5-7 mm when fully opened.
Comparison of lirf with other varieties of liriope muscari: ‘LIRJ’ differs from the variety ‘Evergreen Giant’ (unpatented) as it has a narrow-medium leaf width (mean 7.5 mm), very dense foliage and lighter purple violet colored flowers corresponding to purple violet (RHS 82B). whereas ‘Evergreen Giant’ has a medium leaf width (8-10 mm), a weak density foliage and lighter looking purple violet colored flowers (corresponding to RHS 82D). ‘LIRJ’ has a plant height of 60-65 cm whereas ‘Evergreen Giant’ has a plant height of 70-75 cm. ‘LIRJ’ has a very thick leaf thickness (mean 0.80 mm) creating a strong leaf whereas ‘Evergreen Giant’ has a medium leaf thickness (mean 0.60 mm). ‘LIRJ’ has flowers borne in clusters of 3 along the whole peduncle whereas ‘Evergreen Giant’ has flowers borne in clusters of 4 towards the base changing to clusters of 3 towards the apex. ‘LIRJ’ differs from the variety ‘Big Blue’ (unpatented) as it has a narrow-medium leaf width (mean 7.5 mm), very dense foliage and lighter purple violet colored flowers (corresponding to RHS 82D) whereas ‘Big Blue’ has a broad leaf width (mean 12.1 mm) and medium density foliage and darker looking violet colored flowers (corresponding to RHS 83C). ‘LIRJ’ differs from the variety ‘Bigun’ (U.S. Plant Pat. No. 15,474), as it has a narrow-medium leaf width (mean 7.5 mm), an absence of leaf twisting and purple violet colored flowers (corresponding to RHS 82D) whereas ‘Bigun’ has a broad leaf width (to 18 mm), leaf twisting present and flower color corresponding to violet blue (RHS 92A). ‘LIRJ’ differs from the variety ‘Royal Purple’ (unpatented) as it has a narrow-medium leaf width (mean 7.5 mm), strong shoot density and an inflorescence displaying flowers within the foliage whereas ‘Royal Purple’ has a broad leaf width (mean 12 mm), weak shoot density and an inflorescence displaying flowers slightly above the foliage. The combination of its medium-tall plant height combined with narrow-medium leaf width, very thick leaf thickness, dense foliage and a purple violet flower color makes ‘LIRJ’ a desirable ornamental plant suited for mass production for pot and landscape use.
Asexual reproduction: After its initial discovery, ‘LIRJ’ was transplanted into a 140 mm pot for further trials and testing. After divisions were made for several subsequent generations, ‘LIRJ’ was observed to retain color and size characteristics that were noted in the original ‘LIRJ’ seedling. Divisions of ‘LIRJ’ root within 2 to 3 weeks. ‘LIRJ’ was divided into several pots for further evaluation. Potting mix used contained 50% sand and 50% peat. Plants were divided into 90 mm×50 mm×50 mm tubes. ‘LIRJ’ has been propagated in vitro using micropropagation and has been observed to reproduce from generation to generation in a uniform and stable manner maintaining original color and size characteristics.
Environmental tolerances: ‘LIRJ’ has typical shade tolerance for the species and performed the same as the variety ‘Evergreen Giant’ in garden trials at Clarendon, NSW, Australia. The winter hardiness of ‘LIRJ’ is at least to zone 5A in the United States, and evaluation of winter hardiness is ongoing. ‘LIRJ’ has drought tolerance similar to the variety ‘Evergreen Giant’ with moderate to good recovery with watering after severe wilting. ‘LIRJ’ survived and grew well over 2 years in garden trials in sandy and clay soils, but also tolerates heavy, clay-type soils at Clarendon, NSW, Australia.
Disease resistance: ‘LIRJ’ has typical resistance to fungal and bacterial pathogens compared to other similar Liriope muscari cultivars as demonstrated in garden trials with survival and growth vigor similar to the variety ‘Evergreen Giant’ at Clarendon, NSW, Australia.

That which is claimed is:

1. A new and distinct variety of Liriope muscari plant named ‘LIRJ’, substantially as described and illustrated herein.
